(19) United States
(12) Patent Application Publication
(10) Pub. No.: US 2022/0079990 A1
(43) Pub. Date: Mar. 17, 2022

(54) METHODS AND COMPOSITIONS FOR TREATING SKIN AND HAIR DISORDERS

(71) Applicant: DIRECT BIOLOGICS LLC, Austin, TX (US)

(72) Inventors: Timothy Alexander MOSELEY, Carlsbad, CA (US); Kenneth Allen PETTINE, Fort Collins, CO (US)

(21) Appl. No.: 17/418,342

(22) PCT Filed: Dec. 26, 2019
(86) PCT No.: PCT/US2019/068615

§ 371 Date: Jun. 25, 2021

Related U.S. Application Data
(60) Provisional application No. 62/787,672, filed on Jan. 2, 2019.
(60) Provisional application No. 62/785,072, filed on Dec. 26, 2018.
(60) Provisional application No. 62/785,155, filed on Dec. 26, 2018.
(60) Provisional application No. 62/785,126, filed on Dec. 26, 2018.

(51) Int. Cl. A61K 35/28; A61K 35/36; A61K 38/18; A61Q 5/00; A61K 8/98; A61K 8/64

(52) U.S. Cl. CPC A61K 35/28; A61K 35/36; A61K 38/18; A61K 8/981; A61K 8/985; A61K 8/64; A61Q 5/00

(57) ABSTRACT

Disclosed are treatment mesenchymal stem cell growth factor compositions and methods of their use to treat skin disorders, hair greying, baldness, and erectile dysfunction as well as their use in skin color/ethnic specific cosmetic and skin treatments. In some aspects, the treatment target SNPs specific to the hair color, skin color, skin type, hair type, race, ethnicity, and/or response to testosterone.

### I. BACKGROUND

Humans share 99.9% of the exact same genetics, meaning that differences between humans is only contained in 0.1% of an individual's genetics. The small differences are contained in single nucleotide polymorphisms (SNPs). It is estimated that 6 to 20 of these SNPs are responsible for all the differences observed in skin, eye, and hair color as well as baldness. Thus, the epidermis and dermis of the skin are anatomically, physiologically, and biologically different depending on skin color and ethnicity and responsible for why some people develop grey hair at an early age and some people's hair never turns grey. Similarly, Male pattern baldness is hereditary and appears to be linked to an abnormal reaction of the hair follicle to a byproduct of testosterone. These small differences are contained in single nucleotide polymorphisms (SNPs). Recent studies have discovered that very specific SNP profiles can accurately predict who will develop hair loss and who will not. This is true in males and females. Hair loss can start as early as adolescence, and it affects two thirds of men by age 35 and about 85% of men by the age of 50. This is also a common problem in women. Similarly, specific SNPs have been identified in erectile dysfunction.

Currently, there are few, if any, skin products that exist that are specific to a specific skin color or ethnicity as well as few, if any hair products that account for genetic factors responsible for greying hair or baldness. Similarly, there are no products that address genetic underpinnings associated with erectile dysfunction. Thus, conventional skin and hair products and erectile dysfunction treatments do not adequately address the biological differences found in the dermis of each skin or hair color.

Therefore, what is needed are new dermatological and cosmetic treatments, and a methods of making such treatment, and methods of treatment, that are specific to a person's skin color, hair color, baldness, erectile dysfunction, and specific country or ethnicity, the treatment comprising targeted SNPs of a specific skin color, specific hair color, baldness, and/or erectile dysfunction found in the DNA of an mesenchymal stem cell (MSC), keratinocyte, or melanocyte from a representative donor.

### II. SUMMARY

Disclosed are methods and compositions related to mesenchymal stem cell growth factor compositions and methods of their use to treat skin disorders, hair greying, baldness, and erectile dysfunction as well as their use in skin color/ethnic specific cosmetic and skin treatments.

In one aspect, disclosed herein are methods for creating treatment composition for treating, inhibiting, reducing, preventing, and/or reversing a hair (such as for example, hair greying, baldness (including, but not limited to baldness caused by male pattern baldness, androgenetic alopecia, alopecia areata, cicatricial alopecia, telogen effluvium, and/or female pattern baldness), and/or skin discoloration or disorder (such as, for example, vitiligo, melasma, strawberry nevus, and/or rosacea) and/or erectile dysfunction the method comprising: identifying the body characteristics (such as, for example, skin color, skin type, ethnicity, race, hair type, and/or hair color) of an end user; obtaining mesenchymal skin cells (MSCs) from a targeted donor having the same body characteristics (such as, for example, skin color, skin type, ethnicity, race, hair type, and/or hair color) as the end user but with a single nucleotide polymorphism (SNP) profile that indicates the donor will never experience a hair or skin disorder and/or erectile dysfunction and can be gender, race, and ethnicity specific; preparing an MSC, keratinocyte, and melanocyte growth factor powder preparation from the obtained MSCs, wherein the MSC preparation comprises at least one member selected from the group consisting of cells or cell conditioned media cultured under normal hyperoxic culturing conditions and cells cultured under harsh wound healing conditions; and adding the powder preparation to a base; wherein the treatment composition comprises a member selected from the group consisting of a topical composition and an injectable composition. In one aspect, the treatment composition corrects skin discoloration, stimulates the activation of hair follicles to promote hair growth, stimulates the production of hair pigmentation, or promotes proper erectile function. In one aspect, the SNP comprises one or more SNPs present in one or more genes encoding Interleukin 18 (IL-18), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Plasminogen Activator Inhibitor-1 (PAI-1), Osteopontin (OPN), Insulin, Insulin growth factor binding protein four (IGF-BP4), Tumor Necrosis Factor (TNF) receptor (TNF-R), Neuregulin-1 B1 (NRG1-B1), Urokinase type Plasminogen Activator Receptor (uPAR), Ectodysplasin A2 receptor (XEDAR), follistatin, and/or activated leukocyte cell adhesion molecule (ALCAM). In one aspect, the SNP comprises the IL-18 SNP rs1946518 (−607C>A) and/or rs187238 (−137G>C).

Also disclosed herein are treatment compositions for treating, inhibiting, reducing, preventing, or reversing a hair disorder, skin disorder, and/or erectile dysfunction, the composition comprising: a composition base; and a mesenchymal stem cell (MSC), keratinocyte, and/or melanocyte growth factor powder preparation derived from a donor having the same body characteristics (such as, for example, skin color, skin type, ethnicity, race, hair type, and/or hair color) as an end user but with a single nucleotide polymorphism (SNP) profile that indicates the donor will never have the hair disorder, skin disorder, and/or erectile dysfunction and can be gender, race, and ethnicity specific, wherein the MSC preparation comprises at least one member selected from the group consisting of cells or cell conditioned media cultured under normal hyperoxic culturing conditions and cells cultured under harsh wound healing conditions.

In one aspect, the SNP of the treatment compositions comprises one or more SNPs present in one or more genes encoding Interleukin 18 (IL-18), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Plasminogen Activator Inhibitor-1 (PAI-1), Osteopontin (OPN), Insulin, Insulin growth factor binding protein four (IGF-BP4), Tumor Necrosis Factor (TNF) receptor (TNF-R), Neuregulin-1 B1 (NRG1-B1), Urokinase type Plasminogen Activator Receptor (uPAR), Ectodysplasin A2 receptor (XEDAR), follistatin, and/or activated leukocyte cell adhesion molecule (ALCAM). In one aspect, the SNP comprises the IL-18 SNP rs1946518 (−607C>A) and/or rs187238 (−137G>C).

Also disclosed herein are methods of treating a skin disorder, erectile dysfunction, or a hair disorder in a subject comprising administering to the subject any of the treatment compositions of any preceding aspect.

In one aspect, disclosed herein are treatment compositions of any preceding aspect, wherein the hair disorder comprises baldness (such as, for example baldness comprises male pattern baldness, androgenetic alopecia, alopecia areata, cicatricial alopecia, telogen effluvium, and/or female pattern baldness) or grey hair.

Also disclosed herein are methods for creating a skin treatment composition for a specific skin color, race, type, or ethnicity, the method comprising: identifying the skin color, type, race, and ethnicity of an end user; obtaining mesenchymal skin cells (MSCs) from a targeted donor having the same skin color, type, race, and ethnicity as the end user based on the donor's appropriate single nucleotide polymorphism panel; preparing an MSC, keratinocyte, and melanocyte growth factor powder preparation from the obtained MSCs, wherein the MSC preparation comprises at least one member selected from the group consisting of cells or cell conditioned media cultured under normal hyperoxic culturing conditions and cells cultured under harsh wound healing conditions; and adding the powder preparation to a cosmetic base.

In one aspect, disclosed herein are skin treatment compositions for a specific skin color, race, type, or ethnicity, the composition comprising: a composition base; and a mesenchymal stem cell (MSC), keratinocyte, and/or melanocyte growth factor powder preparation derived from a donor having the same skin color, type race, and ethnicity as an end user, wherein the MSC preparation comprises at least one member selected from the group consisting of cells or cell conditioned media cultured under normal hyperoxic culturing conditions and cells cultured under harsh wound healing conditions.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the effect of the disclosed MSC compositions on wrinkles.

FIGS. 2A, 2B, 2C, 2D, and 2E show the effect of injection of the disclosed MSC compositions on baldness. FIG. 2A shows the ability of an injection comprising MSC exosomes and platelet rich plasma (1:4 ratio) to stimulate hair growth in a subject with male pattern baldness 6 weeks after treatment. FIG. 2B shows the effect of a 1:4 dilution of the disclosed MSC compositions in saline to treat androgenetic alopecia one month following treatment. 0.1 mL was administered for each injection approximately 1 cm apart. FIG. 2C shows the effect of the MSC composition in a 1:1 dilution in saline on male pattern baldness 1 week after treatment. For this treatment 0.1 mL was injected to the affected area approximately 0.5 cm apart. FIG. 2D shows the effect of the MSC composition on male pattern baldness 6 weeks after treatment. FIG. 2E shows the MSC composition in a 1:5 dilution in saline on male pattern baldness 2 weeks after treatment. For this treatment 0.1 mL was injected to the affected area approximately 0.5 cm apart.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “subject” is defined herein to include animals such as mammals, including, but not limited to, primates (e.g., humans), cows, horses, pigs, sheep, goats, dogs, cats, rabbits, rats, mice and the like. In some embodiments, the subject is a human.

“Administration” to a subject includes any route of introducing or delivering to a subject an agent. Administration can be carried out by any suitable route, including oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Controlled release” or “sustained release” refers to release of an agent from a given dosage form in a controlled fashion in order to achieve the desired pharmacokinetic profile in vivo. An aspect of “controlled release” agent delivery is the ability to manipulate the formulation and/or dosage form in order to establish the desired kinetics of agent release.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

A “decrease” can refer to any change that results in a smaller gene expression, protein production, amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The terms “prevent,” “preventing,” “prevention,” and grammatical variations thereof as used herein, refer to a method of partially or completely delaying or precluding the onset or recurrence of a disease and/or one or more of its attendant symptoms or barring a subject from acquiring or reacquiring a disease or reducing a subject's risk of acquiring or reacquiring a disease or one or more of its attendant symptoms.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain (i.e., nociception) relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular treatment composition (such as a composition comprising growth factors, exosomes, or proteins derives from MSC) is disclosed and discussed and a number of modifications that can be made to a number of molecules including the treatment composition (such as a composition comprising growth factors, exosomes, or proteins derives from MSC) are discussed, specifically contemplated is each and every combination and permutation of treatment composition (such as a composition comprising growth factors, exosomes, or proteins derives from MSC) and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

In one aspect, disclosed herein are treatment compositions specific to a subject's body characteristics (such as, for example, skin color, skin type, ethnicity, race, hair type, and/or hair color) for treating, inhibiting, reducing, preventing and/or reversing baldness, hair greying, erectile dysfunction, and/or skin disorders. As disclosed herein, the treatment composition can comprise concentrated growth factors, exosomes, extracellular proteins, proteoglycans, cytokines, chemokines, proteins, and peptides derived from MSCs or similar fibroblast-like cells, keratinocytes or melanocytes, wherein the cells may be obtained from bone marrow, adipose (fat) stromal vascular fraction (SVF), bone, or other tissue sources before or after cell expansion. In some aspect, the treatment composition can further comprise stem cell factors (SCF).

In embodiments, the treatment composition may comprise a base, and an MSC, keratinocyte, and/or melanocyte growth factor powder preparation, wherein the MSC preparation (MSC/K/M/Prep) may include at least one member selected from the group consisting of cells or cell conditioned media cultured under normal hyperoxic culturing conditions and cells cultured under harsh wound healing conditions. Hyperoxic culturing conditions may be defined as about 21%, wherein about 21% may be 21%±5%, oxygen with serum supplements and oxygen, while wound healing conditions may be defined as about 1 to about 5% oxygen in the presence of inflammatory cytokines, angiogenic factors, and/or reduced glucose.

The MSC/K/M/Prep may comprise either conditioned media or lysate from cell culture expanded MSCs, keratinocytes, or melanocytes. In some embodiments, the composition may further comprise from about 0.01 to about 10 wt. % of a cell-free medium conditioned by growth of MSC/K/M/PREP or lineage cells, wherein the cells are cultured under normal hyperoxic culturing conditions or under wound healing conditions.

The MSC/K/M/PREP conditioned media, lysates, and derived products or combinations thereof, optionally with other active ingredients, may be dissolved, mixed, or suspended in a mixture of emulsifying lanolin alcohols, waxes, and oils or a mixture of petrolatum or mineral oil, a quaternary ammonium compound, a fatty alcohol, and a fatty ester emollient, or lotions that are substantially similar in composition.

The base of the composition of the present disclosure may be any suitable or desired base, such as a lotion, a cream, a pigment, a serum, an oil, a gel, a hydrogel, a powder, a foundation, a facial mask, a lip care product, a hair car product, a hair car product, a skin cleanser, an exfoliant, an ointment, or the like. Alternatively, the base may comprise a material suitable for injection directly into the dermis.

In embodiments, the base may comprise a lotion comprising a mixture of emulsifying lanolin alcohols, waxes, and oils or a mixture of petrolatum or mineral oil, a quaternary ammonium compound, a fatty alcohol, and a fatty ester emollient. Alternatively, the base may comprise a cream comprising a mixture of emulsifying lanolin alcohols, water, petrolatum, glycerin, isostearyl palmitate, butylene glycol, glyceryl stearate, or a mixture thereof.

In some embodiments, the cosmetic base may be a carrier that may contain, for example, about 1 to about 20 wt. % of a humectant, about 0.1 to about 10 wt. % of a thickener and water. Alternatively, the carrier may comprise about 70 to about 99 wt. % of a surfactant, and about 0 to about 20 wt. % of a fat. The carrier may alternatively comprise about 80 to 99.9% of a thickener; about 5 to about 15% of a surfactant, about 2 to about 15% of a humectant, about 0 to about 80% of an oil, very small (<2%) amounts of preservative, coloring agent and/or perfume, and water if desired.

In embodiments, the composition may further comprise a penetration enhancer to improve epidermal penetration of the bioactive substance. Suitable penetration enhancers may include dimethyl sulfoxide (DMSO), DMSO-like compounds, ethanolic compounds, pyroglutamic acid esters, and the like. The composition may also include a sunscreen, anti-acne agents, anticellulite agents, and other additional components.

The composition may be filter-sterilized or concentrated. Moreover, the composition may be free from non-human animal products or may be derived from animal sources.

While the composition is described above as including MSCs, the use of other fibroblast-like cells is envisioned. The product may contain keratinocytes or melanocytes. The MSCs may be derived from multiple sources such as bone marrow stroma, adipose, blood, dermis, periosteum, bone, and other tissues. In embodiments, the MSCs may be derived from the patient to which the composition will be applied (autologous) or derived from another individual (allogeneic). The MSCs/K/M/PREP may be culture expanded to collect the conditioned media or to increase the quantity of cells for the lysate or used freshly prior to incorporation into the composition of the present disclosure.

Producing the treatment composition of the present disclosure may include first identifying the target consumer's hair characteristics, sourcing MSC's from an individual with similar hair characteristics, and using the sourced MSC's to create a topical and injectable treatment to be applied to the scalp or the dermis in areas affected by miniaturization, wherein the treatment will stimulate hair follicles for the production of hair growth.

As such, the final treatment composition may be customized for the end user based on hair characteristics. To use the hair and scalp treatment composition of the present disclosure, a user may simply apply the composition topically to the scalp. Alternatively, the composition may be injected directly into the dermis in areas affected by miniaturization. Administration can be as little as 0.1 mL to as much as 100 mL as appropriate for the given indication. For example, the administered dose can be 0.1, 0.2, 0.25, 0.3, 0.4, 0.5, 0.6, 0.7, 0.75, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 mL in total single administration or aliquoted over multiple injections. For example, a single 1 mL volume can be dispersed in 0.1 mL injections. When multiple injections are given, the volume of each injection can be 0.1, 0.2, 0.25, 0.3, 0.4, 0.5, 0.6, 0.7, 0.75, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0, 6, 7, 8, 9, 10 mL. Injections can be made as a single site of injection or over 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 600, 700, 800, 900, or 1000 separate injections. Where multiple injections are used in an effected area, the distance between injection can be 0.1, 0.2, 0.25, 0.3, 0.4, 0.5, 0.6, 0.7, 0.75, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0, 6, 7, 8, 9, or 10 cm apart. While desired, it is understood and herein contemplated that a single treatment administration may not be sufficient to achieve the desired therapeutic results. Accordingly, administration can occur at a single time or 2, 3, 4, 5, 6, 7, 8, 910, 11, 12, 13, 14, 15, 16, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 600, 700, 800, 900, or 1000 separate times for the life of the host or duration of the treatment. When multiple administrations are made, administration can occur one time every 6, 12, 18, 24, 36, 48, 60, 72 hours, 4, 5, 6, 7, 8, 9, 10, 11, 12, 13, 14, 15, 16, 17, 18, 19, 20, 21, 22, 23, 24, 25, 26, 27, 28, 29, 30, 31, 35, 36, 42, 49, 56, 58, 59, 60, 61, 62 days, 9, 10, 11, 12, 13, 14, 15, 16 weeks, 5, 6, 7, 8, 9, 10, 11, 12, 18, 24 months, 3, 4, 5, 6, 7, 8, 9, or 10 years.

It is understood and herein contemplated that the concentrated growth factors, exosomes, extracellular proteins, proteoglycans, cytokines, chemokines, proteins, and peptides derived from MSCs or similar fibroblast-like cells, keratinocytes or melanocytes used in the disclosed treatment compositions can be diluted to reach an administered dose. Diluents can be any suitable substance including but not limited to saline or any pharmaceutical based carrier or excipient disclosed herein. The dilution of the growth factors, exosomes, extracellular proteins, proteoglycans, cytokines, chemokines, proteins, and peptides derived from MSCs or similar: 1fibroblast-like cells, keratinocytes or melanocytes can be 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50 or 1:100.

1. Skin Color

Currently, there are few, if any, skin products that exist that are specific to a specific skin color or ethnicity. Thus, conventional skin products do not adequately address the biological differences found in the dermis of each skin color or specific race.

As noted above, the base may comprise a lotion comprising a mixture of emulsifying lanolin alcohols, waxes, and oils or a mixture of petrolatum or mineral oil, a quaternary ammonium compound, a fatty alcohol, and a fatty ester emollient. Alternatively, the base may comprise a cream comprising a mixture of emulsifying lanolin alcohols, water, petrolatum, glycerin, isostearyl palmitate, butylene glycol, glyceryl stearate, or a mixture thereof.

In yet a further embodiment, the composition of the present disclosure may comprise an eye lash treatment and, thus, the cosmetic base may comprise a mixture of mineral oils, linseed oil, castor, eucalyptus, lanolin, beeswax, or a mixture thereof.

In some aspects, the base can be a cosmetic base and comprise any known ingredients typically found in the cosmetic and cosmeceutical fields, such as oils, waxes or other standard fatty substances, or conventional gelling agents and/or thickeners; emulsifiers; moisturizing agents; emollients; sunscreens; hydrophilic or lipophilic active agents, such as ceramides; agents for combating free radicals; bactericides; sequestering agents; preservatives; basifying or acidifying agents; fragrances; surfactants; fillers; natural products or extracts of natural product, such as aloe or green tea extract; vitamins; or coloring materials. The amounts of the ingredients in the cosmetic base may be dependent on the desired effect.

The cosmetic composition of the present disclosure may provide skin tissue rejuvenation, augmentation, and improved or restored skin tissue. The composition may also be used in the treatment of burns or other wounds as a wound healing enhancement. In embodiments, the composition may also be used to treat erectile dysfunction and vaginal atrophy. Some embodiments do not require the use of commonly used dermal fillers and carriers, such and hyaluronic acid and the like. However, these ingredients may be included. Moreover, embodiments of the composition may not require the inclusion of a growth factor, such as insulin, insulin-like growth factors, thyroid hormones, fibroblast growth factors, estrogen, retinoic acid, and the like. The composition may also not require the inclusion of adipocytes.

The cosmetic composition of the present disclosure may be applied topically to the skin. any area of the skin may be treated, such as the face, the neck, the hands, or any other desired part of the body. In some embodiments, the composition of the present disclosure may be pre-loaded onto a bandage or other dressing, which may be applied to the skin.

As mentioned above, producing the skin treatment composition of the present disclosure may include first identifying the desired skin type, color, race, or ethnicity and obtaining MSCs from a targeted donor having the same skin type, color, race, and ethnicity. As such, the final skin treatment composition may be customized for the end user, based on skin type, color, race, and ethnicity.

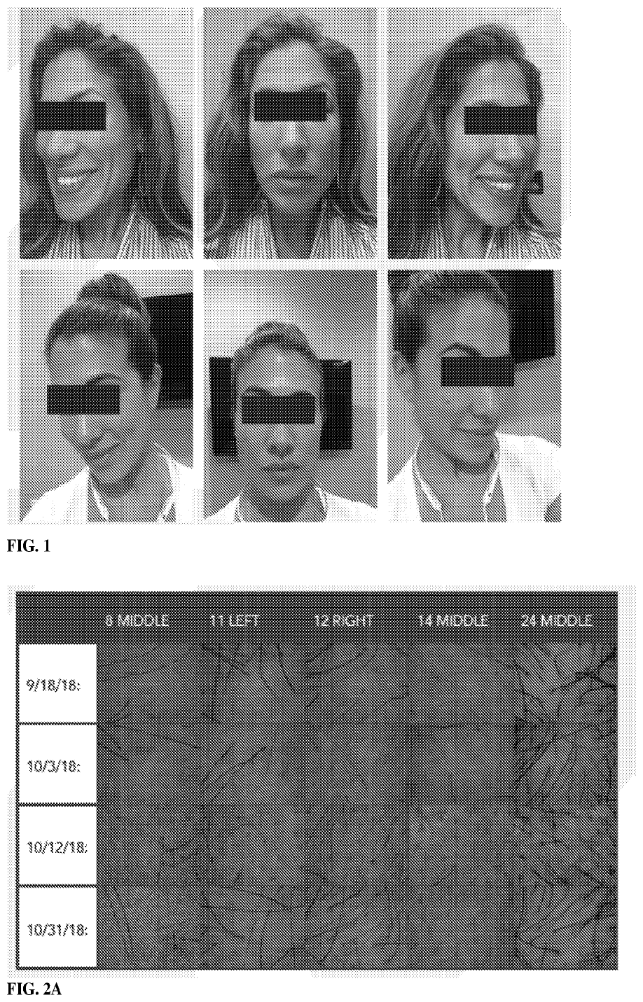

For example, wrinkles in the skin can be treated by injecting the compositions disclosed herein into the site of wrinkles as shown in FIG. 1. There, the MSC comprising composition was diluted at a 1:10 ratio in saline (1 mL of composition to 9 mL of saline) and 0.5 mL was injected into each wrinkle. Within 10 days, the patient had already shown improvement.

Accordingly, disclosed herein are disclosed herein are methods for creating treatment composition for treating, inhibiting, reducing, preventing, and/or reversing a skin discoloration or disorder (such as, for example, vitiligo, melasma, strawberry nevus, and/or rosacea) the method comprising: identifying the body characteristics (such as, for example, skin color, skin type, ethnicity, race, hair type, and/or hair color) of an end user; obtaining mesenchymal skin cells (MSCs) from a targeted donor having the same body characteristics (such as, for example, skin color, skin type, ethnicity, race, hair type, and/or hair color) as the end user but with a single nucleotide polymorphism (SNP) profile that indicates the donor will never experience the skin disorder or discoloration and can be gender, race, and ethnicity specific; preparing an MSC, keratinocyte, and melanocyte growth factor powder preparation from the obtained MSCs, wherein the MSC preparation comprises at least one member selected from the group consisting of cells or cell conditioned media cultured under normal hyperoxic culturing conditions and cells cultured under harsh wound healing conditions; and adding the powder preparation to a base; wherein the treatment composition comprises a member selected from the group consisting of a topical composition and an injectable composition.

Also disclosed herein are treatment compositions for treating, inhibiting, reducing, preventing, or reversing a skin disorder or discoloration (such as, for example, vitiligo, melasma, strawberry nevus, and/or rosacea) the composition comprising: a composition base; and a mesenchymal stem cell (MSC), keratinocyte, and/or melanocyte growth factor powder preparation derived from a donor having the same body characteristics (such as, for example, skin color, skin type, ethnicity, race, hair type, and/or hair color) as an end user but with a single nucleotide polymorphism (SNP) profile that indicates the donor will never have the skin disorder and can be gender, race, and ethnicity specific, wherein the MSC preparation comprises at least one member selected from the group consisting of cells or cell conditioned media cultured under normal hyperoxic culturing conditions and cells cultured under harsh wound healing conditions.

Also disclosed herein are methods of treating a skin disorder in a subject comprising administering to the subject any of the treatment compositions disclosed herein.

Also disclosed herein are methods for creating a skin treatment composition for a specific skin color, race, type, or ethnicity, the method comprising: identifying the body characteristics (such as, for example, skin color, skin type, ethnicity, race, hair type, and/or hair color) of an end user; obtaining mesenchymal skin cells (MSCs) from a targeted donor having the same body characteristics as the end user based on the donor's appropriate single nucleotide polymorphism panel; preparing an MSC, keratinocyte, and melanocyte growth factor powder preparation from the obtained MSCs, wherein the MSC preparation comprises at least one member selected from the group consisting of cells or cell conditioned media cultured under normal hyperoxic culturing conditions and cells cultured under harsh wound healing conditions; and adding the powder preparation to a cosmetic base.

In one aspect, disclosed herein are skin treatment compositions for a specific skin color, race, type, or ethnicity, the composition comprising: a composition base; and a mesenchymal stem cell (MSC), keratinocyte, and/or melanocyte growth factor powder preparation derived from a donor having the same skin color, type race, and ethnicity as an end user, wherein the MSC preparation comprises at least one member selected from the group consisting of cells or cell conditioned media cultured under normal hyperoxic culturing conditions and cells cultured under harsh wound healing conditions.

In one aspect, the SNP of the treatment compositions comprises one or more SNPs present in one or more genes encoding Interleukin 18 (IL-18), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Plasminogen Activator Inhibitor-1 (PAI-1), Osteopontin (OPN), Insulin, Insulin growth factor binding protein four (IGF-BP4), Tumor Necrosis Factor (TNF) receptor (TNF-R), Neuregulin-1 B1 (NRG1-B1), Urokinase type Plasminogen Activator Receptor (uPAR), Ectodysplasin A2 receptor (XEDAR), follistatin, and/or activated leukocyte cell adhesion molecule (ALCAM). In one aspect, the SNP comprises the IL-18 SNP rs1946518 (−607C>A) and/or rs187238 (−137G>C).

2. Grey Hair

Stem cell factor (SCF) expression in the hair matrix identifies immediate antecedents of hair shaft structural cells, and transcription factor KROX20 marks a sub lineage of epithelial cells differentiating toward hair shaft progenitor cells. Stem cell factor (SCF, also known as KIT ligand or Steel factor) is a growth factor that regulates multiple physiological homeostatic events, including the maintenance of hematopoietic stem cells, mast cells, and melanocytes. The critical role of SCF and its receptor, KIT, for hair pigmentation has been supported by the absence of hair pigment in several SCF- and KIT-deprived animals. It is apparent that melanocytes are the relevant target cells in this type of hypopigmentation, as they are the melanin-producing cells as well as the predominant KIT-expressing cell in the hair follicle (HF). The sources of SCF that support melanocytic activity in the HF appear to be dermal fibroblasts.

KROX20 (also known as EGR2) is a zinc finger transcription factor that is expressed in subpopulations of hair follicle (HF) cells. However, the role of the KROX20-expressing cells in HF development is not known. It has been reported that the transcription factor KROX20 identifies a sub lineage of HF epithelial cells toward the differentiation of hair shaft during HF morphogenesis. Observations show that dermal fibroblasts are a necessary source of SCF for the maintenance of melanocytes in the hair matrix to produce hair pigmentation, as evidenced by the complete loss of pigment when SCF is deleted in KROX20 lineage cells.

Studies have shown that depletion of SCF in the hair shaft progenitor cells results in loss of hair pigmentation. In addition, melanocytes are the specialized melanin-producing cells as well as the predominant KIT-expressing cells in the HF. Therefore, melanocytes mediate SCF signaling to hair pigmentation, and SCF is necessary for melanocytic activity and hair pigmentation. This finding demonstrates that loss of SCF in the hair shaft progenitor cells influences only the melanocytes, highlighting the upper HF matrix as a critical niche where noncell-autonomous SCF/KIT signaling regulates hair pigmentation. It also points to the critical role of follicular epithelial SCF in controlling the fate of melanocytes in their final maturation without affecting their previous differentiation and migration. SCF production in fibroblasts and keratinocytes appears to regulate the generation of hair pigmentation by melanocytes.

Fibroblasts secrete numerous melanogenic factors, such as stem cell factor (SCF), hepatocyte growth factor (HGF), keratinocyte growth factor (KGF), corticotrophin-releasing hormone (CRH), endothelin-I (ET-I), interferon-y (IFN-γ), and interleukin-I Melanocytes are activated through multiple paracrine factors secreted from adjacent cells, such as keratinocytes and fibroblasts. The paracrine factors bind to the specific receptors expressed on melanocytes and then accelerate signal transductions to initiate melanin production

While the above connections with hair pigmentation are known, currently, there is no existing product to prevent or treat grey hair at a cellular level. Rather, grey hair is conventionally addressed by dying the grey hair to match the desired hair color.

Accordingly, disclosed herein are methods for creating treatment composition for treating, inhibiting, reducing, preventing, and/or reversing a hair greying the method comprising: identifying the body characteristics (such as, for example, skin color, skin type, ethnicity, race, hair type, and/or hair color) of an end user; obtaining mesenchymal skin cells (MSCs) from a targeted donor having the same body characteristics (such as, for example, skin color, skin type, ethnicity, race, hair type, and/or hair color) as the end user but with a single nucleotide polymorphism (SNP) profile that indicates the donor will never experience grey hair and can be gender, race, and ethnicity specific; preparing an MSC, keratinocyte, and melanocyte growth factor powder preparation from the obtained MSCs, wherein the MSC preparation comprises at least one member selected from the group consisting of cells or cell conditioned media cultured under normal hyperoxic culturing conditions and cells cultured under harsh wound healing conditions; and adding the powder preparation to a base; wherein the treatment composition comprises a member selected from the group consisting of a topical composition and an injectable composition. In one aspect, the treatment composition corrects hair pigmentation and/or stimulates the production of hair pigmentation. In one aspect, the SNP of the methods of creating a treatment compositions comprises one or more SNPs present in one or more genes encoding Interleukin 18 (IL-18), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Plasminogen Activator Inhibitor-1 (PAI-1), Osteopontin (OPN), Insulin, Insulin growth factor binding protein four (IGF-BP4), Tumor Necrosis Factor (TNF) receptor (TNF-R), Neuregulin-1 B1 (NRG1-B1), Urokinase type Plasminogen Activator Receptor (uPAR), Ectodysplasin A2 receptor (XEDAR), follistatin, and/or activated leukocyte cell adhesion molecule (ALCAM). In one aspect, the SNP comprises the IL-18 SNP rs1946518 (−607C>A) and/or rs187238 (−137G>C).

Also disclosed herein are treatment compositions for treating, inhibiting, reducing, preventing, or reversing a hair greying, the composition comprising: a composition base; and a mesenchymal stem cell (MSC), keratinocyte, and/or melanocyte growth factor powder preparation derived from a donor having the same body characteristics (such as, for example, skin color, skin type, ethnicity, race, hair type, and/or hair color) as an end user but with a single nucleotide polymorphism (SNP) profile that indicates the donor will never have grey hair and can be gender, race, and ethnicity specific, wherein the MSC preparation comprises at least one member selected from the group consisting of cells or cell conditioned media cultured under normal hyperoxic culturing conditions and cells cultured under harsh wound healing conditions.

In one aspect, the SNP of the treatment compositions comprises one or more SNPs present in one or more genes encoding Interleukin 18 (IL-18), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Plasminogen Activator Inhibitor-1 (PAI-1), Osteopontin (OPN), Insulin, Insulin growth factor binding protein four (IGF-BP4), Tumor Necrosis Factor (TNF) receptor (TNF-R), Neuregulin-1 B1 (NRG1-B1), Urokinase type Plasminogen Activator Receptor (uPAR), Ectodysplasin A2 receptor (XEDAR), follistatin, and/or activated leukocyte cell adhesion molecule (ALCAM). In one aspect, the SNP comprises the IL-18 SNP rs1946518 (−607C>A) and/or rs187238 (−137G>C).

Also disclosed herein are methods of treating a grey hair in a subject comprising administering to the subject any of the treatment compositions disclosed herein.

3. Baldness

Hair is made of a protein called keratin. Hair sits in a hair follicle, and at the base of the follicle are stem cells, called follicular epithelial stem cells, that stimulated the production of new hair. The average head has over 100,000 hair follicle and each follicle cycles producing hair and then resting. The cycle of producing hair and resting is normal and results in always having a full head of hair.

Baldness occurs because more and more hair follicles are in the resting stage or shrink until they never produce new hair. Because of genetics, men destined to be bald have hair follicles that are overly sensitive to the actions of dihydrotestosterone (DHT), which is a byproduct of testosterone. DHT binds to hair follicles and causes them to shrink. More and more hair follicles shrink until they product no hair, which causes areas to develop hair thinning and eventual baldness. This process is called miniaturization.

Currently, only two drugs are approved by the FDA to treat hair loss—minoxidil and finasteride. Minoxidil is available as an over-the-counter topical, but most experts agree that minoxidil is a relatively marginal effective drug in the fight against hair loss and has zero effect on the process of miniaturization. Thus, its benefits are temporary. Finasteride works by inhibiting testosterone become DHT, but its side effects include erectile dysfunction and libido and ejaculation disorders. Low level laser therapy has been shown to stimulate hair growth, in both men and women, but has no effect on miniaturization.

Hair transplantation is another method for treating baldness and involves harvesting follicles from the back of the head that are DHT resistant and transplanting them to bald areas. However, this does not stimulate new growth as the patient still has the same amount of hair, it is just redistributed more evenly around the scalp. What is needed is a composition and method for preventing and treating baldness, wherein the method and composition promotes the stimulation of the follicular epithelial stem cells and mesenchymal stem cells in the follicle to result in activated follicles to produce hair and prevent miniaturization.

Some embodiments of the present disclosure include a method for producing a composition for preventing and treating baldness comprising preparing a concentration of mesenchymal stem cell (MSC) exosomes and secretomes from a targeted donor with specific SNP hair characteristics that indicate the donor will never experience hair loss. The donor may be gender specific. The method may include identifying the target donor by SNP hair characteristics, culturing MSCs from the target donor to create a cultured media under either normal hyperoxic culturing conditions or harsh wound healing hypoxic conditions; creating a powder from the cultured media; and combining the powder with a cream or lotion base, wherein the final product may be applied to the dermis in areas affected by miniaturization. Application may be either topical or injectable and may stimulate the activation of hair follicles to promote hair growth, particularly in areas affected by miniaturization or by direct injection into the dermis.

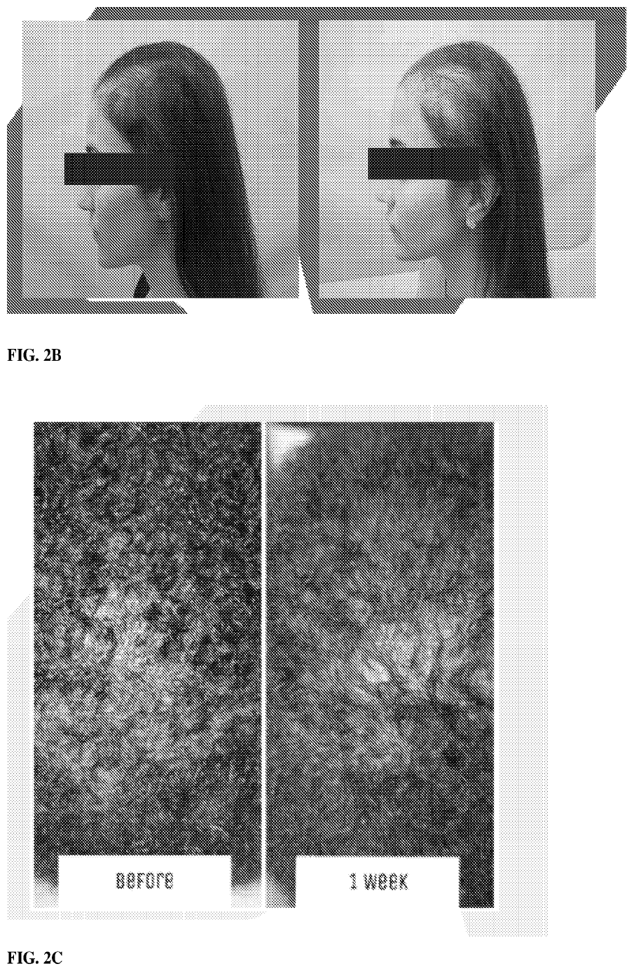

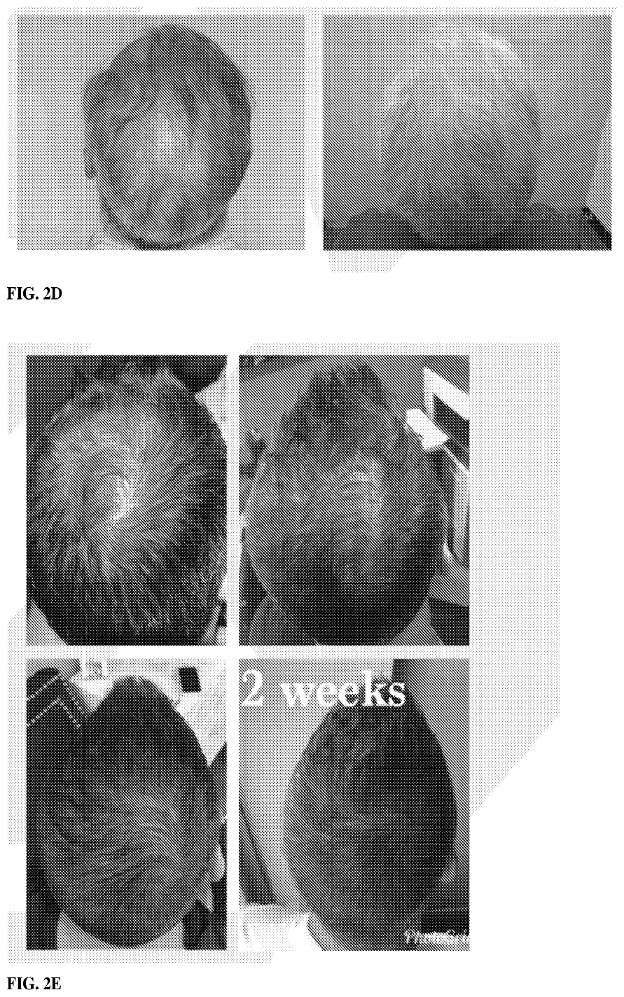

When injected into the scalp of the affected areas of subjects with male pattern baldness or androgenetic alopecia, successful hair regrowth was observed in as little as 2 weeks post treatment (see FIGS. 2A-2E).

A proteomic assessment of exosome suspensions generated using methods and parameters described herein was performed to characterize the molecular compositions that contribute to clinically relevant efficacy of the described invention. The proteomic assessments utilized commercially available antibody arrays manufactured by RayBio Tech, Norcross, Ga., USA). Concentration measurements were made of 230 different proteins known to either be secreted, transported or present on the external surface of cell membranes (within the extracellular microenvironment). Proteins measured present at physiologically relevant concentrations in duplicate test samples and which were found to be supported as relevant to hair restoration and colorization are listed in Table 1. A physiologically relevant concentration was deemed to be a mean concentration of ≥1 pg/mL. Literature surveys of proteins with average concentrations greater than 50 ug/mL were performed to identify most likely candidate molecules to support a clinical effect. The list in Table 1 should Not be considered to be a comprehensive or exhaustive list of individual proteins found within the invention that are relevant to efficacious effects.

TABLE 1
Survey of Invention Protein Content.

| | | Exosome | Exosome |
|---|---|---|---|
| | Mean | Suspension | Suspension |
| | Concentration | Sample 1 | Sample 2 |
| Protein Identity | [pg/mL] | [pg/mL] | [pg/mL] |
| IL-18 | 50033.50 | 29801.41 | 70265.59 |
| PDGF Rb | 10178.43 | 10036.71 | 10320.16 |
| IGFBP-4 | 5840.14 | 3878.93 | 7801.34 |
| TIMP-2 | 1708.44 | 2121.46 | 1295.42 |
| TIMP-1 | 684.26 | 734.23 | 634.29 |
| IL-23 | 372.10 | 393.96 | 350.24 |
| Activin A | 454.81 | 378.14 | 531.48 |
| ICAM-2 | 193.61 | 365.61 | 21.60 |
| XEDAR | 226.68 | 317.96 | 135.40 |
| OPN | 254.12 | 252.85 | 255.38 |
| TNF RI | 126.27 | 136.16 | 116.37 |
| Follistatin | 95.93 | 129.93 | 61.93 |

Proteins are organized from highest to lowest mean concentration. Proteins expressed at concentrations greater than the arbitrary value of 50 ng/mL were surveyed in the medical literature using PubMed search engine to identify studies that provide evidence for potential effects of this invention. Proteins in the invention supported in the scientific literature to play a role in hair follicle regeneration are listed and described below.

IL18 was the most concentrated protein present in the exosome suspension samples. It's primary function involves regulation of the innate immune response within the skin, specifically, stimulating interferon gamma production and activating dermal natural killer and TH1 T-cells. Too much IL18 is associated with onset autoimmune diseases. The literature survey provides evidence for a role in hair growth. Two single nucleotide polymorphisms (SNPs) found within IL18 gene sequence are associated with development of the organ specific autoimmune disease, alopecia areata (AA). Two specific SNPs, rs1946518 (−607C>A) and rs187238 (−137G>C) polymorphisms are associated with alopecia areata disease. Celik et al concluded that IL-18 rs187238 and rs1946518 SNPs may be the cause of the AA susceptibility.¹ IL18 may play a role in observed irregular interactions between perifollicular mast cells and CD8+ cells. These interactions may disrupt the normal hair growth cycle within the follicles. By histology analysis, IL18 and its receptor are found within skin keratinocytes, and within the outer sheath cells of hair follicles. Two possible effects of IL18 action to initiate hair growth observed when the invention is applied to the dermis is that the sequence of the IL18 from this donor does not have the disease associated sequences and changes IL18 signaling levels to a non-disease state level. Alternatively, evidence of IL18 isoforms exists including a predominant form in sera that may function as an autocrine inhibitor. The form of IL18 in this invention may be an inhibitory isoform that reduces IL18 signaling and restores an appropriate the innate immune response to an appropriate level.

It has been demonstrated that induction of anagen phase using conditioned media from hypoxic adipose derived mesenchymal stem/stromal cell populations. PDGF-receptor B was a major protein detected and. PDGF may stimulate dermal papillae proliferation through the PDGF receptor. Exosomic introduction of PDGF-Receptor B into membranes of telogenic dermal papillae cells may enable signaling via PDGF that initiates anagenic hair growth. Genetic variants of PDGFR-B are associated with hair loss in Penttinen Syndrome.

Insulin growth factors are agonists for hair growth. IGF binding proteins regulate IGF activities by binding to IGF. Binding to the various IGF-BPs can modify IGF activity and provide additional specificity of IGF activity. IGF-BP4, along with IGF-BP3 and IGF-BP5 are expressed inhuman hair follicle dermal papillae and serve to regulate IGF activity within the hair follicle.

Normal hair growth is cyclic. Each hair follicle undergoes extracellular remodeling throughout each cycle. TIMP-1 and TIMP-2 play critical roles in regulating proteolytic activity of collagenases and other proteases involved remodeling of extracellular matrix in and around the hair follicle. Additional TIMPs provided by the invention may restore balance to levels of proteases altered by inflammatory disease states associated with alopecia.

Interleukin twenty-three is found in hair follicles at a higher level during Alopecia Areata; however, it's exact function within the hair follicles is still unknown. Like other cytokines, IL23 may have multiple functions depending on isoform, and receptors expressed by different cell types. Additional IL23 provided by the invention could provide an autocrine inhibitory signal that helps decrease the inflammatory condition resulting in hair growth suppression 88. The activins are members of the TGF-B signaling pathway and are critical for the initial formation of hair follicles during development, and they play a key role in epidermal/mesenchymal interactions required during hair organogenesis. Subsequently, in combination with follistatin, activin is an important regulator of the hair cycle.

ICAMs function to connect and create a barrier between cells. The hair follicle is an immune-privileged micro-organ. ICAMs establish physical barriers that establish that immune privilege environment. In alopecia areata, the immune privileged environment is disrupted enabling development of an autoimmune response to antigens within the melanocytes that provide color to the hair. By providing ICAM-2, the invention may enable reestablishment of the immune privileged micro-environment

Osteopontin is expressed by outer hair follicle sheath cells. Osteopontin is proteolytically cleaved in vivo to generate peptide signaling molecules that regulate FGF-7 production by the outer root sheath keratinocytes. The osteopontin derived peptide appears to inhibit FGF-7 synthesis which in turns slows hair growth. Osteopontin therefore may be an important regulator of the hair growth cycle.

EDAR and XEDAR bind ectodysplasin family members Eda A1 and Eda A2. XEDAR activates NFKB signaling path and is associated with signaling during hair follicle morphogenesis. Murine gene knockout studies of this signaling pathway leads to malformation of hair follicles.

During hair follicle regeneration following dermal wound healing, the presence of macrophage signaling is critical to initiate new hair follicle formation. Lgr5+ cells are the first stem cells activated during telogen-anagen transition in hair cycle. Macrophages are shown to activate these cells to proliferate via TNF signaling. Introduction of TNFR-1 via uptake of the invention into Lgr5+ cells in the hair follicle bulb may increase the sensitivity of these cells to respond to TNF signals and initiate hair follicle formation.

Collectively, the protein characterization data provided for the invention demonstrates feasibility that prominent protein elements may function to reinitiate dormant hair follicles via multiple modes of action that includes but is not limited to modification or resolution of inhibitory disease and inflammation states, activation of dermal, melanocyte, and keratinocyte cell proliferation, and induction of follicular cell differentiation and extracellular matrix production.

Accordingly, disclosed herein are methods for creating treatment composition for treating, inhibiting, reducing, preventing, and/or reversing baldness (including, but not limited to baldness caused by male pattern baldness, androgenetic alopecia, alopecia areata, cicatricial alopecia, telogen effluvium, and/or female pattern baldness) the method comprising: identifying the body characteristics (such as, for example, skin color, skin type, ethnicity, race, hair type, and/or hair color) of an end user; obtaining mesenchymal skin cells (MSCs) from a targeted donor having the same body characteristics (such as, for example, skin color, skin type, ethnicity, race, hair type, and/or hair color) as the end user but with a single nucleotide polymorphism (SNP) profile that indicates the donor will never experience baldness and can be gender, race, and ethnicity specific; preparing an MSC, keratinocyte, and melanocyte growth factor powder preparation from the obtained MSCs, wherein the MSC preparation comprises at least one member selected from the group consisting of cells or cell conditioned media cultured under normal hyperoxic culturing conditions and cells cultured under harsh wound healing conditions; and adding the powder preparation to a base; wherein the treatment composition comprises a member selected from the group consisting of a topical composition and an injectable composition. In one aspect, the treatment composition stimulates the activation of hair follicles to promote hair growth. In one aspect, the SNP of the methods of creating a treatment composition comprises one or more SNPs present in one or more genes encoding Interleukin 18 (IL-18), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Plasminogen Activator Inhibitor-1 (PAI-1), Osteopontin (OPN), Insulin, Insulin growth factor binding protein four (IGF-BP4), Tumor Necrosis Factor (TNF) receptor (TNF-R), Neuregulin-1 B1 (NRG1-B1), Urokinase type Plasminogen Activator Receptor (uPAR), Ectodysplasin A2 receptor (XEDAR), follistatin, and/or activated leukocyte cell adhesion molecule (ALCAM). In one aspect, the SNP comprises the IL-18 SNP rs1946518 (−607C>A) and/or rs187238 (−137G>C).

Also disclosed herein are treatment compositions for treating, inhibiting, reducing, preventing, or reversing a baldness (including, but not limited to baldness caused by male pattern baldness, androgenetic alopecia, alopecia areata, cicatricial alopecia, telogen effluvium, and/or female pattern baldness), the composition comprising: a composition base; and a mesenchymal stem cell (MSC), keratinocyte, and/or melanocyte growth factor powder preparation derived from a donor having the same body characteristics (such as, for example, skin color, skin type, ethnicity, race, hair type, and/or hair color) as an end user but with a single nucleotide polymorphism (SNP) profile that indicates the donor will never be bald and can be gender, race, and ethnicity specific, wherein the MSC preparation comprises at least one member selected from the group consisting of cells or cell conditioned media cultured under normal hyperoxic culturing conditions and cells cultured under harsh wound healing conditions.

In one aspect, the SNP of the treatment compositions comprises one or more SNPs present in one or more genes encoding Interleukin 18 (IL-18), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Plasminogen Activator Inhibitor-1 (PAI-1), Osteopontin (OPN), Insulin, Insulin growth factor binding protein four (IGF-BP4), Tumor Necrosis Factor (TNF) receptor (TNF-R), Neuregulin-1 B1 (NRG1-B1), Urokinase type Plasminogen Activator Receptor (uPAR), Ectodysplasin A2 receptor (XEDAR), follistatin, and/or activated leukocyte cell adhesion molecule (ALCAM). In one aspect, the SNP comprises the IL-18 SNP rs1946518 (−607C>A) and/or rs187238 (−137G>C).

Also disclosed herein are methods of treating a baldness (including, but not limited to baldness caused by male pattern baldness, androgenetic alopecia, alopecia areata, cicatricial alopecia, telogen effluvium, and/or female pattern baldness) in a subject comprising administering to the subject any of the treatment compositions disclosed herein.

4. Erectile Dysfunction

A proteomic assessment of exosome suspensions generated using methods and parameters described herein was performed to characterize the molecular compositions that contribute to clinically relevant efficacy of the described invention. The proteomic assessments utilized commercially available antibody arrays manufactured by RayBio Tech, Norcross, Ga., USA). Concentration measurements were made of 230 different proteins known to either be secreted, transported or present on the external surface of cell membranes (within the extracellular microenvironment). Proteins measured present at physiologically relevant concentrations in duplicate test samples and that were supported in the scientific literature to be potentially efficacious for treatment of erectile dysfunction are listed in Table 2. A physiologically relevant concentration was deemed to be a mean concentration of ≥1 pg/mL. Literature surveys of proteins with average concentrations greater than 50 ug/mL were performed to identify most likely candidate molecules to support a clinical effect.

TABLE 2
Survey of Invention Protein Content.

| | | Exosome | Exosome |
|---|---|---|---|
| | Mean | Suspension | Suspension |
| | Concentration | Sample 1 | Sample 2 |
| Protein Identity | [pg/mL] | [pg/mL] | [pg/mL] |
| IL-18 | 50033.50 | 29801.41 | 70265.59 |
| PDGF Rb | 10178.43 | 10036.71 | 10320.16 |
| TIMP-1 | 684.26 | 734.23 | 634.29 |
| PAI-1 | 400.41 | 434.72 | 366.10 |
| OPN | 254.12 | 252.85 | 255.38 |
| Insulin | 177.33 | 164.70 | 189.95 |
| TNF RI | 126.27 | 136.16 | 116.37 |
| NRG1-b1 | 44.46 | 86.66 | 2.26 |
| uPAR | 32.41 | 61.69 | 3.14 |
| ALCAM | 54.35 | 48.73 | 59.96 |

Proteins are organized from highest to lowest mean concentration. Proteins expressed at concentrations greater than the arbitrary value of 50 ng/mL were surveyed in the medical literature using PubMed search engine to identify studies that provide evidence for potential effects of this invention. Bolded font indicates that literature evidence for protein relevance in one or more of the listed clinical indications was established.

Protein content within this invention with capacity to positively effect vascular formation and function, somatic or autonomic innervation and function, or smooth muscle cell physiology may have beneficial effects for erectile dysfunction. Research literature support potential effects of various protein components of this invention to improve or resolve elements of erectile dysfunction pathophysiology.

IL18 was the most concentrated protein present in the exosome suspension samples. It's primary function involves regulation of the innate immune response within the skin, specifically, stimulating interferon gamma production and activating dermal natural killer and TH1 T-cells. Too much IL18 is associated with onset autoimmune diseases. Elevated levels of IL-18 were observed in a population of obese men with erectile dysfunction. Evidence of IL18 isoforms exists including a predominant form in sera that may function as an autocrine inhibitor. The form of IL18 in this invention may be an inhibitory isoform that reduces IL18 proinflammatory signaling and modulates immune responses to an appropriate level. In one aspect, the SNP comprises the IL-18 SNP rs1946518 (−607C>A) and/or rs187238 (−137G>C).

Platelet rich plasma has been used to treat ED. Platelet Derived Growth Factor (PDGF) receptor (PDGFR) signaling has been shown to be important in regulating the differentiation and contractility of corpus cavernosum smooth muscle cells, and regulating connective tissue remodeling. PDGFR-B integration into CCSMC cell membranes could increase sensitivity or rate of response to PDGF signals that improve penile smooth muscle cell function.

A common cause of erectile dysfunction is accumulation of collagenous fibrotic tissue in the penis. Tissue Inhibitors of Metalloprotease (TIMP)-1 (TIMP-1) and TIMP-2 play critical roles in regulating proteolytic activity of collagenases and other proteases. TIMP-1 from mesenchymal stem cells was shown to restore erection function in a rat model of erectile dysfunction.

Myoendothelial junctions are important to establish anatomical compartmentalization required to enable penile erection. In a PAI-1 (Plasminogen Activator Inhibitor-1) knockout mouse model of Erectile dysfunction, recombinant PAI-1 restored the number of myoendothelial junctions in the corporal tissue and also induced a significant decrease in time to maximal corporal pressures. Myoendothelial junctions were similarly identified in human corporal tissue. These results suggest a critical role for myoendothelial junctions in erectile pathophysiology and therapies like this invention may restore myoendothelial junction numbers in the corporal tissue and provide a novel therapy for erectile dysfunction.

Osteopontin (OPN) is commonly associated with bone matrix formation; however, since it's initial characterization in bone, osteopontin has been shown to have additional functionality in other cell types. For example, it is shown to have anti-apoptosis activity in cell types such as oligodendrocyte progenitors. In a cavernosal crushed nerve model of ED in rats an increased amount of osteopontin was observed. In this scenario osteopontin may be participating in regulation of apoptosis of myelin generating cells near the nerve and play a role in cell saving and enabling faster repair of the damaged tissue.

TNF functions to regulate cell growth and death in numerous different tissue types, TNFR-1 (Tumor Necrosis Factor Receptor-1) is typically described as initiator of apoptotic signaling when be to TNF-alpha as a homodimer; however, TNF receptors can interact with other members of TNF receptor super family of proteins and may become decoy molecules that down regulate the activity of TNF. In this capacity TNFR-1 may function as an inhibitor of chronic inflammatory signaling involved with some forms of erectile dysfunction etiology.

Other neuregulin family molecules (GGF2, for example) have been shown to have a neuro-protective effect in prostectomy related nerve injury that leads to erectile dysfunction. NRG1-B1 (Neuregulin-1 B1) similarly may support neuron growth and restoration of damaged nerves resultant from acute injury related onset of erectile dysfunction since in rat models of neuropathic pain it is shown to alter neuronal function and significantly reduce pain.

Neurons release uPAR (Urokinase type Plasminogen Activator Receptor) during the recovery phase from an ischemic injury, and astrocytes, axonal boutons and dendritic spines recruit uPAR to their plasma membrane. The binding of uPA to uPAR promotes the repair of synapses damaged by an ischemic injury. Uptake of uPA receptors into recipient neurons could increase synaptic connectivity of parasympathetic and sympathetic neuronal circuitry that is malfunctioning in some forms of erectile dysfunction.

ALCAM plays a role within immune, endothelial and neuronal cell physiology. It has been shown to be important to maintain the blood brain barrier as well as play a role in dorsal root ganglion neuron axon extension and cell growth. ALCAM has been identified by one study to be the closest gene to two different SNPs associated with type I diabetes related erectile dysfunction. ALCAM from the invention could serve to provide signals to endothelial and neuronal cells or modify the immune status of penile tissue in erectile dysfunction that result in restoration of tissue integrity and neuro-connectivity.

Collectively, the protein characterization data provided for the invention demonstrates feasibility that prominent protein elements may function to initiate repair or decrease negative signaling events that result in erectile dysfunction. Proteins of this invention may act directly on neurons and smooth muscle cells involved in regulating vascular pressure, and fluid inflow and outflow as well as indirectly addressing external disease conditions that affect the penis anatomy and physiology.

Accordingly, disclosed herein are methods for creating treatment composition for treating, inhibiting, reducing, preventing, and/or reversing erectile dysfunction the method comprising: identifying the body characteristics (such as, for example, skin color, skin type, ethnicity, race, hair type, and/or hair color) of an end user; obtaining mesenchymal skin cells (MSCs) from a targeted donor having the same body characteristics (such as, for example, skin color, skin type, ethnicity, race, hair type, and/or hair color) as the end user but with a single nucleotide polymorphism (SNP) profile that indicates the donor will never experience erectile dysfunction and can be gender, race, and ethnicity specific; preparing an MSC, keratinocyte, and melanocyte growth factor powder preparation from the obtained MSCs, wherein the MSC preparation comprises at least one member selected from the group consisting of cells or cell conditioned media cultured under normal hyperoxic culturing conditions and cells cultured under harsh wound healing conditions; and adding the powder preparation to a base; wherein the treatment composition comprises a member selected from the group consisting of a topical composition and an injectable composition. In one aspect, the treatment composition promotes proper erectile function.

Also disclosed herein are treatment compositions for treating, inhibiting, reducing, preventing, or reversing erectile dysfunction, the composition comprising: a composition base; and a mesenchymal stem cell (MSC), keratinocyte, and/or melanocyte growth factor powder preparation derived from a donor having the same body characteristics (such as, for example, skin color, skin type, ethnicity, race, hair type, and/or hair color) as an end user but with a single nucleotide polymorphism (SNP) profile that indicates the donor will never have erectile dysfunction and can be gender, race, and ethnicity specific, wherein the MSC preparation comprises at least one member selected from the group consisting of cells or cell conditioned media cultured under normal hyperoxic culturing conditions and cells cultured under harsh wound healing conditions. In one aspect, the SNP of the methods of creating a treatment composition disclosed herein comprises one or more SNPs present in one or more genes encoding Interleukin 18 (IL-18), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Plasminogen Activator Inhibitor-1 (PAI-1), Osteopontin (OPN), Insulin, Insulin growth factor binding protein four (IGF-BP4), Tumor Necrosis Factor (TNF) receptor (TNF-R), Neuregulin-1 B1 (NRG1-B1), Urokinase type Plasminogen Activator Receptor (uPAR), Ectodysplasin A2 receptor (XEDAR), follistatin, and/or activated leukocyte cell adhesion molecule (ALCAM). In one aspect, the SNP comprises the IL-18 SNP rs1946518 (−607C>A) and/or rs187238 (−137G>C).

In one aspect, the SNP of the treatment compositions comprises one or more SNPs present in one or more genes encoding Interleukin 18 (IL-18), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Plasminogen Activator Inhibitor-1 (PAI-1), Osteopontin (OPN), Insulin, Insulin growth factor binding protein four (IGF-BP4), Tumor Necrosis Factor (TNF) receptor (TNF-R), Neuregulin-1 B1 (NRG1-B1), Urokinase type Plasminogen Activator Receptor (uPAR), Ectodysplasin A2 receptor (XEDAR), follistatin, and/or activated leukocyte cell adhesion molecule (ALCAM). In one aspect, the SNP comprises the IL-18 SNP rs1946518 (−607C>A) and/or rs187238 (−137G>C).

Also disclosed herein are methods of treating erectile dysfunction in a subject comprising administering to the subject any of the treatment compositions disclosed herein.

5. Mesenchymal Stem Cells

As noted throughout, the treatment compositions disclosed herein can utilize exosomes and/or growth factors derived from mesenchymal stem cells (MSCs). While existing autogenous and allogeneic MSCs contained within bone marrow concentrate or adipose-derived stromal vascular fraction (SVF) or various post-natal products from umbilical cord, placenta or amnion, expanded MSC cultures are currently being used to treat wounds, orthopedic pathology, and spine pathology; the existing treatments do not contain large amounts of MSC secretomes (including, but not limited to growth factors, cytokines, chemokines, exosomes, extracellular vesicles, and/or extracts). Additionally, despite evidence in the art that treatments comprising stem cells (including injectable treatments) can help prevent aging and treat scarring, uneven pigmentation, existing skin products, such as creams, lotions, serums, make-up, and the like, while including ingredients that potentially help treat and strengthen the skin, other topical products do not penetrate the epidermis and more importantly do not include human MSCs, or MSC-derived growth factors and proteins. In fact, prior to the present disclosure an active MSC growth factor product that can be used for these applications has not been developed. Thus, in one aspect, disclosed herein are MSC secretome compositions (including, but not limited to MSC growth factor, MSC exosome, MSC extracts and/or extracellular vesicle comprising compositions) for use in the treatment of wounds, orthopedic disorders, orthopedic injuries, ophthalmology, spinal injury, or spinal disorders, said treatment compositions comprising (i) a growth factor powdered additive comprising a mesenchymal stem cell (MSC) derived preparation and (ii) a pharmaceutically acceptable carrier.

As noted above, MSC are multipotent cells that have the ability to differentiate into a multitude of cell types including myocytes, chondrocytes, adipocytes, and osteoblasts. Typically, these cells can be found in the placenta, umbilical cord blood, adipose tissue, bone marrow, or amniotic fluid, including perivascular tissue. As used herein, “MSC” refers to non-terminally differentiated cells including but not limited to multipotential stem cell, multipotential stromal cell, stromal vascular cells, pericytes, perivascular cells, stromal cells, pluripotent cells, multipotent cells, adipose-derived fibroblast-like cells, adipose-derived stromal vascular fraction, adipose-derived MSC, bone marrow-derived fibroblast-like cells, bone marrow-derived stromal vascular fraction, bone marrow-derived MSC, tissue-derived fibroblast-like cells, adult stem cells, adult stromal cells, keratinocytes, and/or melanocytes.

It has been long recognized that MSC, in addition to their differentiation potential, have the immunomodulatory abilities resulting in the expression of many different cytokines and growth factors. As used herein, a “MSC preparation” or “MSC secretome composition” refers to a composition comprising MSC growth factors, MSC exosomes, extracellular vesicles, or acellular extracts of MSCs or MSC lysates obtained from human MSCs, fibroblast-like cells, and non-human animal MSCs including, but not limited to MSCs from horses, cows, pigs, sheep, non-human primates, dogs, cats, rabbits, rats, and mice. In embodiments, the MSCs may be derived from the patient to which the composition will be applied (autologous) or derived from another individual (allogeneic). The MSCs may be culture expanded to collect the conditioned media or to increase the quantity of cells for the lysate or used freshly prior to incorporation into the composition of the present disclosure.

The MSC secretome compositions (including, but not limited to MSC growth factor, MSC exosome, MSC extracts and/or extracellular vesicle comprising compositions) may comprise about 0.00001 to about 20 wt. %, such as from about 0.01 to about 10 wt. %, of a mesenchymal stem cell (MSC) extract, MSC exosome, or MSC growth factor preparation. The MSC preparation may comprise either MSC conditioned media or MSC lysate from cell culture expanded MSCs. In some embodiments, the composition may further comprise from about 0.01 to about 10 wt. % of a cell-free medium conditioned by growth of MSCs or MSC lineage cells, wherein the cells are cultured under normal hyperoxyic culturing conditions or under artificial wound healing conditions.

As disclosed herein the MSCs used to produce the disclosed MSC additives (including growth factor secretome composition either frozen or powdered additives) can be selectively stimulated to produce MSC growth factors, secretomes, cytokines, chemokines, mesenchymal stem cell proteins, peptides, glycosaminoglycans, extracellular matrix (ECM), proteoglycans, secretomes, and exosomes. As used herein, MSC growth factors include but are not limited to prostaglandin E2 (PGE2), transforming growth factor (β1 (TGF-β1), hepatocyte growth factor (HGF), stromal cell derived factor-1 (SDF-1), nitric oxide, indoleamine 2,3-dioxygenase, interleukin-4 (IL-4), IL-6, interleukin-10 (IL-10), IL-1 receptor antagonist and soluble TNF-α receptor, insulin-like growth factors, fibroblast growth factors (FGF) 1-23 (especially, FGF1 and FGF2), bone morphogenetic proteins (BMPs) 1-15, epidermal growth factor (EGF), transforming growth factor-a (TGF-a) macrophage-stimulating protein (MSP), platelet derived growth factor (PLGF), vascular endothelial growth factor (VEGF), macrophage colony stimulating factor (M-CSF), insulin, granulocyte colony stimulating factor (G-CSF), granulocyte macrophage colony stimulating factor (GM-CSF), as well as hormones including estrogen, and thyroid hormones.

In one aspect, the MSC preparation (such as, for example, a MSC secretome composition) comprises MSC growth factors, MSC exosomes, and/or cellular extracts of MSCs or MSC lysates obtained from MSCs cultured under standard hyperoxyic culturing conditions (for example, 21% oxygen) or MSCs cultured under artificial wound healing conditions (such as, for example, 0.1% to about 5% oxygen in the presence of inflammatory cytokines, angiogenic factors, and reduced glucose).

As disclosed herein artificial wound healing conditions simulate growth conditions in real wounds where there is a reduction in nutrient supply and reduction of waste removal that is usually caused by a disruption in local blood circulation. This creates a harsh environment for cells until new blood vessels are created and blood circulation is restored. Accordingly, artificial wound healing conditions used to culture MSCs can include one or more of the following growth conditions reduction in glucose availability, reduction in oxygen tension, reduction in pH, and increased temperature.

In one aspect, the glucose availability can be reduced relative to normal control. Modified culture media to reduce glucose, but not damage the cells can be between 0 and 50% reduction in glucose, more preferably between about 5% and 40% reduction in glucose. For example, MSC artificial wound healing culture conditions can comprise glucose reduction of about 1, 2, 3, 4, 5, 6, 7, 8 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% such as a glucose reduction from about 5% to about 15%, from about 10% to about 20%, from about 15% to about 25%, from about 20% to about 30%, or from about 25% to about 35%.

In one aspect, oxygen tension can be reduced to oxygen levels to hypoxic conditions. Normal atmospheric oxygen is approximately 21% and any reduction is considered hypoxic. Thus, in one aspect, MSCs can be cultured at between 0.0% and 20.9% oxygen, from about 0.1% to about 0.5% oxygen, from about 0.1% to about 2.0%, from about 0.1% to about 5.0% oxygen, from about 0.5% to 5.0%, from about 1.0% to about 10% oxygen, about 5.0% to about 10.0% oxygen; and from about 10.0% to about 15.0% under artificial wound healing conditions. Preferably during MSC would healing culture conditions oxygen tension is between about 0.5% and 20.5% oxygen, such as, for example, 0, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.7, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, or 20.5% oxygen.

The pH can also be reduced under artificial wound healing conditions. Physiologic pH is maintained very tightly and is usually very close to a neutral pH=7.2±0.2 (7.0-7.4). However, in a wound the acidic environment can have a pH=6.2±0.2 (i.e., a pH from 6.0 to about 6.4). Thus, under artificial wound healing culture conditions, pH can be from about 6.0 to about 7.4, for example, from 6.0 to about 6.4, from about 6.2 to about 6.4, from about 6.2 to about 6.6, from about 6.4 to about 6.6, from about 6.4 to about 6.8, or from about 6.6 to about 7.0, such as 6.0, 6.1, 6.2, 6.3, 6.4, 6.5, 6.6, 6.7, 6.8, 6.9, 7.0, 7.1, 7.2, 7.3 or 7.4.

Under artificial wound healing culture conditions, the temperature of the culture environment may be raised to simulate temperature increases at the site of a wound. Physiologic homeostasis temperature is maintained at 37° C. (98.6° F.). A slight increase or decrease can cause significant changes to cellular metabolism. By increasing the temperature above 37° C. to any temperature up to about 40° C. (104° F.) can create an “feverous” environment. Thus, in on aspect, the artificial wound healing culture conditions for the MSCs can comprise from about 35° C. to about 39° C., from about 35° C. to about 36° C., from about 36° C. to about 37° C., from about 37° C. to about 38° C., from about 38° C. to about 39° C., from about 39° C. to about 40° C. In one aspect, the temperature of the artificial wound healing culture can be 35.0, 35.1, 35.2, 35.3, 36.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, or 40.0° C.

In one aspect, the MSC secretome compositions (including, but not limited to MSC growth factor, MSC exosome, MSC extracts and/or extracellular vesicle comprising compositions) can further comprise a protective coating (such as, for example, a cryoprotectant oligosaccharide and a protein solution) to reduce degradation of the growth factors. It is understood and herein contemplated that the protective coating can be engineered as a polymer. “Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc. In one aspect, the gel matrix can comprise copolymers, block copolymers, diblock copolymers, and/or triblock copolymers.

In one aspect, the protective coating can comprise a biocompatible polymer. In one aspect, biocompatible polymer can be crosslinked. Such polymers can also serve to slowly release the adipose browning agent and/or fat modulating agent into tissue. As used herein biocompatible polymers include, but are not limited to polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly (gly colic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols (PVA), methacrylate PVA(m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers thereof, and blends thereof.

In some embodiments the protective coating comprises carbohydrate construction of monosaccharides as well as carbohydrate polymers such as disaccharides or polysaccharides including but not limited to non-reducing poly or disaccharides as well as any combination thereof. Examples of carbohydrates that can be used in the protective coating comprise Glucose, Aldoses (D-Allose, D-Altrose, D-Mannose, etc.), Glucopyranose, Pentahydroxyhexanal, α-D-Glucopyranosyl-D-glucose, α-D-Glucopyranosyl-dihydrate, Polymer of β-D-Glycopyranosyl units, β-D-Fructofuranosyl α-D-glucopyranoside (anhydrous/dihydrate), β-D-Galactopyranosyl-D-glucose, α-D-Glucopyranosyl-α-D-glucopyranoside (anhydrous/dihydrate), Galactose, Pentoses (Ribose, xylose, lyxose), Dextrose, Dodecacarbon monodecahydrate, Fructose, Sucrose, Lactose, Maltose, Trehalose, Agarose, D-galactosyl-β-(1-4)-anhydro-L-galactosyl, Cellulose, Polymer of β-D-Glycopyranosyl units, and Starch, as well as, Polyhydric alcohols, Polyalcohols, Alditols, Erythritol, Glycitols, Glycerol, Xylitol, and Sorbitol.

In some embodiments the protective coating contains biocompatible and/or biodegradable polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95, 96, 97, 98, or 99 percent acetal pendant groups.

The triblock copolymers disclosed herein comprise a core polymer such as, example, polyethylene glycol (PEG), polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like.

Examples of diblock copolymers that can be used in the protective coatings disclosed herein comprise a polymer such as, example, polyethylene glycol (PEG), polyvinyl acetate, polyvinyl alcohol (PVA), polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA).

In one aspect, the protective coating contains (i.e., the encapsulated, the encapsulated compositions can further comprise lecithin or hydrolyzed lecithin as a carrier or as encapsulation material. As used herein, lecithin and/or hydrolyzed lecithin coatings include coatings comprising phosphatidyl choline, phosphatidyl inositol, phosphatidyl ethanolamine, phosphatidylserine, and phosphatidic acid. Sources of the lecithin can be pnat or animal sources.

In one aspect, any of the polymers, monosaccharides, disaccharides, or polysaccharides used to form the protective coating formed by placing the MSC additive in a encapsulating solution can be at an appropriate concentration for form the protective coating. For example, polymers, monosaccharides, disaccharides, or polysaccharides can be at any concentration between 0.01 mM and 10.0M concentration, for example, from about 0.01M to about 0.1M, from about 0.1 mM to about 1.0M, from about 1.0M to about 10.0M. Exemplary concentrations include 0.01, 0.02, 0.03, 0.04, 0.05, 0.06, 0.07, 0.08, 0.09, 0.1, 0.2, 0.3, 0.4, 0.4, 0.6, 0.7, 0.8, 0.9, 1, 2, 3, 4, 5, 6, 7, 8, 9, 10, 20, 30, 40, 50, 60, 70, 80, 90, 100, 110, 120, 130, 140, 150, 160, 170, 180, 190, 200, 225, 250, 275, 300, 325, 350, 375, 400, 450, 500, 600, 700, 800, 900 mM, 1, 1.1, 1.2, 1.3, 1.4, 1.5, 1.6, 1.7, 1.8, 1.9, 2, 3, 4, 5, 6, 7, 8, 9, 10M. 133. As shown in FIGS. 1 and 2, the exosomses and extracellular vesicles in the disclosed MSC secretome compositions have been produced.

In one aspect, it is understood and herein contemplated that one way to treat a wound is through administration of the MSC secretome compositions (including, but not limited to MSC growth factor, MSC exosome, MSC extracts and/or extracellular vesicle comprising compositions) subcutaneously, intramuscularly, intravenously, topically (such as, for example, through the use of salves, creams, and/or ointments), but also by impregnating stents, sponges, matrixes, scaffolds, bandages, dressing, sutures, grafts, surgical drapes, surgical adhesive, and/or staples with the MSC secretome compositions. Thus, in one aspect, disclosed herein are medicated stents, scaffolds, sponges, matrixes, adhesive bandages, wound dressings, grafts, surgical drapes, sutures, salves, creams, or wound adhesives comprising a therapeutically effective amount of the MSC secretome composition. The MSC secretome compositions (including, but not limited to MSC growth factor, MSC exosome, MSC extracts and/or extracellular vesicle comprising compositions), as noted above, can be administered topically and applied to the face, the neck, the hands, or any other desired part of the body. When applied to an adhesive bandage, wound dressing, grafts, surgical drape, suture, scaffold, matrix, sponge, or stent, the MSC secretome composition can be a applied as a powder.

In one aspect, the MSC secretome compositions (including, but not limited to MSC growth factor, MSC exosome, MSC extracts and/or extracellular vesicle comprising compositions) disclosed herein may comprise any known ingredients typically found in the wound healing fields, such as oils, waxes or other standard fatty substances, or conventional gelling agents and/or thickeners; emulsifiers; moisturizing agents; emollients; sunscreens; hydrophilic or lipophilic active agents, such as ceramides; agents for combating free radicals; bactericides; sequestering agents; preservatives; basifying or acidifying agents; fragrances; surfactants; fillers; natural products or extracts of natural product, such as aloe or green tea extract; vitamins; or coloring materials. Other ingredients that may be combined with the powder may include an antioxidant, which can be selected from a variety of antioxidants. Suitable antioxidants include vitamins, such as Vitamin C (L-Ascorbate, Ascorbate-2 Phosphate magnesium salt, Ascorbyl Palmitate, Tetrahexyldecyl Ascorbate), Vitamin E (Tocotrienol), Vitamin A (retinol, retinal, retinoic acid, provitamin A carotenoids, such as beta-carotene), N-acetyl glucosamine, or other derivatives of glucosamine. Other ingredients may include at least one essential fatty acid, such as Ω-3, Ω-6, and Ω-9 polyunsaturated fatty acids, such as linoleic acid (LA), gamma-linoleic acid (GLA), alpha-linoleic acid (ALA), dihomo-y-linolenic acid (DGLA), arachidonic acid (ARA), and others. The fatty acids may be derived from various sources including evening primrose oil, black currant oil, borage oil, or GLA modified safflower seeds. Other ingredients may include a platelet rich fibrin matrix, at least one ingredient to support ECM production and production of hyaluronic acid, such as N-acetyl glucosamine or other derivatives of glucosamine, ultra-low molecular weight (ULMW) hyaluronic acid, chondroitin sulfate, or keratin sulfate.

It is understood and herein contemplated that the MSC secretome compositions disclosed herein can provide wound healing rejuvenation, augmentation, and improved or restored skin tissue. The composition may also be used as an injectable in the treatment of joint arthritis and degenerated spinal discs. Moreover, embodiments of the composition may not require the inclusion of additional growth factors or hormones, such as insulin, insulin-like growth factors, thyroid hormones, fibroblast growth factors, estrogen, retinoic acid, and the like. In some aspect, the disclosed stem cell growth factor compositions can comprise additional active ingredients including, but not limited to antibiotics, anti-acne agents, liposomes, antioxidants, platelet-rich fibrin matrixes, analgesic, anti-inflammatories, as well as, additional growth factors, such as insulin, insulin-like growth factors, thyroid hormones, fibroblast growth factors, estrogen, retinoic acid, and the like. Such additional active ingredients can be mixed with the stem cell growth factor and extracellular vesicle compositions disclosed herein as well as MSC conditioned media, MSC lystates, and MSC-derived produces and then thawed or dissolved, mixed, or suspended in a mixture of emulsifying lanolin alcohols, waxes, and oils or a mixture of petrolatum or mineral oil, a quaternary ammonium compound, a fatty alcohol, and a fatty ester emollient, or lotions that are substantially similar in composition.

6. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

The preceding examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### C. References

Alam M, Bertolini M, Gherardini J, Keren A, Ponce L, Chéret J, Alenfall J, Dun& P, Nilsson A H, Gilhar A, Paus R. An osteopontin-derived peptide inhibits human hair growth at least in part by decreasing fibroblast growth factor-7 production in outer root sheath keratinocytes. Br J Dermatol. 2019
Aversa A, Basciani S, Visca P, Arizzi M, Gnessi L, Frajese G, Fabbri A. Platelet-derived growth factor (PDGF) and PDGF receptors in rat corpus cavernosum: changes in expression after transient in vivo hypoxia. J Endocrinol. 2001 August; 170(2):395-402.
Batch J A, Mercuri F A, Werther G A. Identification and localization of insulin-like growth factor-binding protein (IGFBP) messenger RNAs in human hair follicle dermal papilla. J Invest Dermatol. 1996 March; 106(3):471-5.
Bertolini M, Zilio F, Rossi A, et al. Abnormal interactions between perifollicular mast cells and CD8+ T-cells may contribute to the pathogenesis of alopecia areata. PLoS ONE. 2014; 9:e94260.
Boraschi D, Dinarello C A. IL-18 in autoimmunity: review. Eur Cytokine Netw. 2006; 17:224-252. Fouad N A, Baraka E A, Hassan W A. Interleukin-18 gene polymorphisms in systemic lupus erythematosus: relation to disease status. Egypt J Immunol. 2014; 21:1-12.
Botchkarev V A, Fessing M Y. Edar signaling in the control of hair follicle development. J Investig Dermatol Symp Proc. 2005 December; 10(3):247-51.
Burnett A L, Sezen S F, Hoke A, Caggiano A O, laci J, Lagoda G, Musicki B, Bella A J. GGF2 is neuroprotective in a rat model of cavernous nerve injury-induced erectile dysfunction. J Sex Med. 2015 April; 12(4):897-905.
C Mazaheri N, Peymani M, Galehdari H, Ghaedi K, Ghoochani A, Kiani-Esfahani A, Nasr-Esfahani M H. Ameliorating Effect of Osteopontin on H(2)O(2)-Induced Apoptosis of Human
Oligodendrocyte Progenitor Cells. Cell Mol Neurobiol. 2018 May; 38(4):891-899.
Carneiro F S, Webb R C, Tostes R C. Emerging role for TNF-α in erectile dysfunction. J Sex Med. 2010 December; 7(12):3823-34.
Celik S D, Ates O. Genetic analysis of interleukin 18 gene polymorphisms in alopecia areata. J Clin Lab Anal. 2018 June; 32(5):e22386.
Chen C C, Murray P J, Jiang T X, Plikus M V, Chang Y T, Lee O K, Widelitz R B, Chuong C M. Regenerative hair waves in aging mice and extra-follicular modulators follistatin, dkkl, and sfrp4. J Invest Dermatol. 2014 August; 134(8):2086-2096.
CHUN HOU, YONG MIAO, XUE WANG, CHAOYUE CHEN, BOJIE LIN, ZHIQI H U Expression of matrix metalloproteinases and tissue inhibitor of matrix metalloproteinases in the hair cycle Exp Ther Med. 2016 July; 12(1): 231-237. Published online 2016 May 9.
Epifanova M V, Chalyi M E, Krasnov A O. [Investigation of mechanisms of action of growth factors of autologous platelet-rich plasma used to treat erectile dysfunction]. Urologiia. 2017 September; (4):46-48. Russian.
Gilhar A. Collapse of immune privilege in alopecia areata: coincidental or substantial? J Invest Dermatol. 2010 November; 130(11):2535-7.
Giugliano F, Esposito K, Di Palo C, Ciotola M, Giugliano G, Marfella R, D'Armiento M, Giugliano D. Erectile dysfunction associates with endothelial dysfunction and raised proinflammatory cytokine levels in obese men. J Endocrinol Invest. 2004 July-August; 27(7):665-9.
Hotaling J M, Waggott D R, Goldberg J, Jarvik G, Paterson A D, Cleary P A, Lachin J, Sarma A, Wessells H; DCCT/EDIC Research Group. Pilot genome-wide association search identifies potential loci for risk of erectile dysfunction in type 1 diabetes using the DCCT/EDIC study cohort. J Urol. 2012 August; 188(2):514-20.
Johnston J J, Sanchez-Contreras M Y, Keppler-Noreuil K M, Sapp J, Crenshaw M, Finch N A, Cormier-Daire V, Rademakers R, Sybert V P, Biesecker L G. A Point Mutation in PDGFRB Causes Autosomal-Dominant Penttinen Syndrome. Am J Hum Genet. 2015 Sep. 3; 97(3):465-74.
Kavoussi P K, Heberlein K, Straub A C, Lowe G J, Oliver J L, Smith R P, Steers W D, Annex B H, Isakson B E, Lysiak J J. Recombinant PAI-1 therapy restores myoendothelial junctions and erectile function in PAI-1-deficient mice. Andrologia. 2015 December; 47(10):1147-52.
Kawabe T T, Rea T J, Flenniken A M, Williams B R, Groppi V E, Buhl A E. Localization of TIMP in cycling mouse hair. Development. 1991 April; 111(4):877-9
Kim S K, Park H J, Chung J H, et al. Association between interleukin 18 polymorphisms and alopecia areata in Koreans. Jlnterferon Cytokine Res. 2014; 34:349-353.
Kim S K, Park H J, Chung J H, et al. Association between interleukin 18 polymorphisms and alopecia areata in Koreans. Jlnterferon Cytokine Res. 2014; 34:349-353.
Koizumi H, Sato-Matsumura K C, Nakamura H, Shida K, Kikkawa S, Matsumoto M, Toyoshima K, Seya T. Distribution of IL-18 and IL-18 receptor in human skin: various forms of IL-18 are produced in keratinocytes. Arch Dermatol Res. 2001 July; 293(7):325-33. PubMed PMID: 11550805.
Lécuyer M A, Saint-Laurent O, Bourbonnière L, Larouche S, Larochelle C, Michel L, Charabati M, Abadier M, Zandee S, Haghayegh Jahromi N, Gowing E, Pittet C, Lyck R, Engelhardt B, Prat A. Dual role of ALCAM in neuroinflammation and blood-brain barrier homeostasis. Proc Natl Acad Sci USA. 2017 Jan. 24; 114(4):E524-E533.
McDowall M, Edwards N M, Jahoda C A, Hynd P I. The role of activins and follistatins in skin and hair follicle development and function. Cytokine Growth Factor Rev. 2008 October December; 19(5-6):415-26.
Park B S, Kim W S, Choi J S, Kim H K, Won J H, Ohkubo F, Fukuoka H. Hair growth stimulated by conditioned medium of adipose-derived stem cells is enhanced by hypoxia: evidence of increased growth factor secretion. Biomed Res. 2010 February; 31(1):27-34
Qian S Q, Qin F, Zhang S, Yang Y, Wei Q, Wang R, Yuan J H. Vacuum therapy prevents corporeal veno-occlusive dysfunction and penile shrinkage in a cavernosal nerve injured rat model. Asian J Androl. 2019 Jun. 25.
Sivalingam S P, Yoon K H, Koh D R, Fong K Y. Single-nucleotide polymorphisms of the interleukin-18 gene promoter region in rheumatoid arthritis patients: protective effect of A A genotype. Tissue Antigens. 2003; 62:498-504.
Suarez-Fariñas M, Ungar B, Noda S, Shroff A, Mansouri Y, Fuentes-Duculan J, Czemik A, Zheng X, Estrada Y D, Xu H, Peng X, Shemer A, Krueger J G, Lebwohl M G, Guttman-Yassky E. Alopecia areata profiling shows TH1, TH2, and IL-23 cytokine activation without parallel TH17/TH22 skewing. J Allergy Clin Immunol. 2015 November; 136(5):1277-87.
Thelen K, Jaehrling S, Spatz J P, Pollerberg G E. Depending on its nano spacing, ALCAM promotes cell attachment and axon growth. PLoS One. 2012; 7(12):e40493.
Vanamee É S, Faustman D L. Structural principles of tumor necrosis factor superfamily signaling. Sci Signal. 2018 Jan. 2; 11(511).
Wang G, Dai D, Chen X, Yuan L, Zhang A, Lu Y, Zhang P. Upregulation of neuregulin-1 reverses signs of neuropathic pain in rats. Int J Clin Exp Pathol. 2014 Aug. 15; 7(9):5916-21.
Wang X, Chen H, Tian R, Zhang Y, Drutskaya M S, Wang C, Ge J, Fan Z, Kong D, Wang X, Cai T, Zhou Y, Wang J, Wang J, Wang S, Qin Z, Jia H, Wu Y, Liu J, Nedospasov S A, Tredget E E, Lin M, Liu J, Jiang Y, Wu Y. Macrophages induce AKT/P-catenin-dependent Lgr5(+) stem cell activation and hair follicle regeneration through TNF. Nat Commun. 2017 Mar. 27; 8:14091.
Yan J F, Huang W J, Zhao J F, Fu H Y, Zhang G Y, Huang X J, Lv B D. The platelet-derived growth factor receptor/STAT3 signaling pathway regulates the phenotypic transition of corpus cavernosum smooth muscle in rats. PLoS One. 2017 Feb. 28; 12(2):e0172191.
Yepes M. Urokinase-type plasminogen activator is a modulator of synaptic plasticity in the central nervous system: implications for neurorepair in the ischemic brain. Neural Regen Res. 2020 April; 15 (4): 620-624.
Zhou F, Hui Y, Xu Y, Lei H, Yang B, Guan R, Gao Z, Xin Z, Hou J. Effects of adipose-derived stem cells plus insulin on erectile function in streptozotocin-induced diabetic rats. Int Urol Nephrol. 2016 May; 48(5):657-69.
Zhou J, Ling J, Wang Y, Shang J, Ping F. Cross-talk between interferon-gamma and interleukin-18 in melanogenesis. J Photochem Photobiol B. 2016. October; 163:133-43.
Zhou J, Shang J, Song J, Ping F. Interleukin-18 augments growth ability of primary human melanocytes by PTEN inactivation through the AKT/NF-κB pathway. Int J Biochem Cell Biol. 2013; 45:308-331.

1. A method for creating treatment composition for treating, inhibiting, reducing, preventing, or reversing a hair or skin disorder or treating erectile dysfunction, the method comprising:
identifying the hair, skin color, skin type, race, ethnicity of an end user;
obtaining mesenchymal skin cells (MSCs) from a targeted donor having the same hair type, color, and/or ethnicity or not susceptible to erectile dysfunction as the end user but with a single nucleotide polymorphism (SNP) profile that indicates the donor will never experience hair disorder, skin disorder, or erectile dysfunction, and can be gender, race, and ethnicity specific;
preparing an MSC, keratinocyte, and melanocyte growth factor powder preparation from the obtained MSCs, wherein the MSC preparation comprises at least one member selected from the group consisting of cells or cell conditioned media cultured under normal hyperoxic culturing conditions and cells cultured under harsh wound healing conditions; and
adding the powder preparation to a base,
wherein the treatment composition comprises a member selected from the group consisting of a topical composition and an injectable composition.

2. The method of claim 1, wherein the hair or skin disorder comprises hair greying, baldness, or skin discoloration.

3. The method of claim 2, wherein the skin discoloration results from vitiligo, melasma, strawberry nevus, or rosacea.

4. The method of claim 3, wherein the treatment composition corrects skin discoloration.

5. The method of claim 2, wherein the baldness comprises male pattern baldness, androgenetic alopecia, alopecia areata, cicatricial alopecia, telogen effluvium, or female pattern baldness.

6. The method of claim 5, wherein the treatment composition stimulates the activation of hair follicles to promote hair growth.

7. The method of claim 2, wherein the treatment composition stimulates the production of hair pigmentation.

8. The method of claim 1, wherein the SNP comprises one or more SNPs present in one or more genes encoding Interleukin 18 (IL-18), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Plasminogen Activator Inhibitor-1 (PAI-1), Osteopontin (OPN), Insulin, Insulin growth factor binding protein four (IGF-BP4), Tumor Necrosis Factor (TNF) receptor (TNF-R), Neuregulin-1 B1 (NRG1-B1), Urokinase type Plasminogen Activator Receptor (uPAR), Ectodysplasin A2 receptor (XEDAR), follistatin, and/or activated leukocyte cell adhesion molecule (ALCAM).

9. The method of claim 1, wherein the SNP comprises the IL-18 SNP rs1946518 (−607C>A) and/or rs187238 (−137G>C).

10. A treatment composition for treating, inhibiting, reducing, preventing, or reversing a hair or skin disorder, the composition comprising:
a composition base; and
a mesenchymal stem cell (MSC), keratinocyte, and/or melanocyte growth factor powder preparation derived from a donor having the same hair type, hair color, hair type, and/or hair color as an end user but with a single nucleotide polymorphism (SNP) profile that indicates the donor will never the hair disorder and/or skin disorder and can be gender, race, and ethnicity specific, wherein the MSC preparation comprises at least one member selected from the group consisting of cells or cell conditioned media cultured under normal hyperoxic culturing conditions and cells cultured under harsh wound healing conditions.

11. The treatment composition of claim 10, wherein the SNP comprises one or more SNPs present in one or more genes encoding Interleukin 18 (IL-18), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Plasminogen Activator Inhibitor-1 (PAI-1), Osteopontin (OPN), Insulin, Insulin growth factor binding protein four (IGF-BP4), Tumor Necrosis Factor (TNF) receptor (TNF-R), Neuregulin-1 B1 (NRG1-B1), Urokinase type Plasminogen Activator Receptor (uPAR), Ectodysplasin A2 receptor (XEDAR), follistatin, and/or activated leukocyte cell adhesion molecule (ALCAM).

12. The treatment composition of claim 10, wherein the SNP comprises the IL-18 SNP rs1946518 (−607C>A) and/or rs187238 (−137G>C)

13. The treatment composition of claim 10, wherein the hair disorder comprises baldness or grey hair.

14. The treatment composition of claim 10, wherein the baldness comprises male pattern baldness, androgenetic alopecia, alopecia areata, cicatricial alopecia, telogen effluvium, or female pattern baldness.

15. A method of treating a hair disorder comprising administering to a subject any of the treatment compositions of claim 10.

16. The treatment composition of claim 10, wherein the skin disorder comprises skin discoloration that results from vitiligo, melasma, strawberry nevus, or rosacea.

17. A method of treating a skin disorder comprising administering to a subject any of the treatment compositions of claim 10.

18. A method for creating a skin treatment composition for a specific skin color, race, type, or ethnicity, the method comprising:
identifying the skin color, type, race, and ethnicity of an end user;
obtaining mesenchymal skin cells (MSCs) from a targeted donor having the same skin color, type, race, and ethnicity as the end user based on the donor's appropriate single nucleotide polymorphism panel;
preparing an MSC, keratinocyte, and melanocyte growth factor powder preparation from the obtained MSCs, wherein the MSC preparation comprises at least one member selected from the group consisting of cells or cell conditioned media cultured under normal hyperoxic culturing conditions and cells cultured under harsh wound healing conditions; and
adding the powder preparation to a cosmetic base.

19. A skin treatment composition for a specific skin color, race, type, or ethnicity, the composition comprising:
a composition base; and
a mesenchymal stem cell (MSC), keratinocyte, and/or melanocyte growth factor powder preparation derived from a donor having the same skin color, type race, and ethnicity as an end user, wherein the MSC preparation comprises at least one member selected from the group consisting of cells or cell conditioned media cultured under normal hyperoxic culturing conditions and cells cultured under harsh wound healing conditions.
